(19) United States
(12) Patent Application Publication
(10) Pub. No.: US 2021/0322376 A1
(43) Pub. Date: Oct. 21, 2021

(54) METHODS FOR REDUCING THE RISK OF DIABETES IN PATIENTS BEING TREATED FOR HIGH CHOLESTEROL-RELATED ILLNESSES

(71) Applicant: Esperion Therapeutics, Inc., Ann Arbor, MI (US)

(72) Inventor: Narendra Dhanraj Lalwani, Ann Arbor, MI (US)

(21) Appl. No.: 17/271,045

(22) PCT Filed: Aug. 26, 2019
(86) PCT No.: PCT/US2019/048184

§ 371 Date: Feb. 24, 2021

Related U.S. Application Data
(60) Provisional application No. 62/751,404, filed on Oct. 26, 2018.
(60) Provisional application No. 62/722,766, filed on Aug. 24, 2018.

(51) Int. Cl. A61K 31/397; A61K 31/194; A61K 45/06; A61P 3/10

(52) U.S. Cl. CPC A61K 31/397; A61P 3/10; A61K 45/06; A61K 31/194

(57) ABSTRACT

Disclosed herein are compositions comprising fixed doses of ETC-1002, ezetimibe, and statin, and methods of treating subjects comprising administering fixed doses of ETC-1002, ezetimibe, and statin. Also disclosed herein are methods for administering fixed doses of ETC-1002 or ezetimibe or both to statin-intolerant patients or patients receiving statin therapy, wherein the administration reduces the likelihood of worsening diabetes in the subject or increasing the likelihood of new onset diabetes in a subject. The methods disclosed herein also include methods of treating hypercholesterolemia and cardiovascular diseases in a subject.

### CROSS-REFERENCE TO RELATED APPLICATIONS

This application claims priority to U.S. Provisional Application No. 62/722,766 filed Aug. 24, 2018 and U.S. Provisional Application No. 62/751,404 filed Oct. 26, 2018, the contents of each of which are incorporated herein by reference.

### BACKGROUND

### Field of the Invention

This application relates to methods and compositions useful for treating diabetes or reducing the risk of diabetic conditions. Low-density lipoprotein cholesterol (LDL-C) and well-established indicator for cardiovascular diseases, as well as a risk factor for diabetes. Similar, hemoglobin A1c (HbA_{1C}) level is a well-known biomarker for diabetes and new-onset diabetes. A common and cornerstone treatment for managing LDL-C level for patients who are either diabetic, at risk of developing new-onset diabetes or at risk of cardiovascular diseases is the administration of statins. However, many patients, for example those with hypercholesterolemia, fail to reduce LDL-C to desired levels with traditional statin therapies. New pharmaceutical drugs have been developed and are effective at reducing cholesterol levels in the human body. Unfortunately, these drugs also induce negative side-effects. Many of the compounds which have been shown to be potent for inhibiting the enzymes of cholesterol biosynthesis are also systemically toxic. Thus, there is a need for new pharmaceutical formulations which are both effective and safe for reducing cholesterol, improving hemoglobin A1c levels, and reducing the risk of developing cardiovascular diseases and diabetic conditions.

### SUMMARY

This application relates to compositions comprising fixed doses of any one of ETC-1002, ezetimibe and statins, and methods for treating or reducing the risk of diabetes comprising the administration of ETC-1002, or ETC-1002 and ezetimibe, in the presence or absence of statin treatment.

ETC-1002 (bempedoic acid) is a typically oral, typically once-daily therapeutic which lowers cholesterol by inhibiting adenosine triphosphate (ATP) citrate lyase (ATPCL). ATPCL is farther upstream than HMG-CoA reductase in the cholesterol biosynthetic pathway.

ETC-1002 lowers low-density lipoprotein cholesterol (LDL-C) by direct inhibition of hepatic adenosine triphosphate citrate lyase, leading to reduced de novo cholesterol synthesis and increased LDL receptor expression. ETC-1002 administered in doses from about 120 mg to about 240 mg daily reduced LDL-C by about 27% to about 43% in phase 2a clinical trials of various hypercholesterolemic populations including patients with type 2 diabetes mellitus and patients with muscle-related statin intolerance.

The general class of “statins” are compounds which lower cholesterol levels in the body by inhibiting the enzyme 3-hydroxy-3-methylglutaryl-coenzyme A (HMG-CoA) reductase and concomitantly, the pathway for synthesizing cholesterol in the liver. Examples of compounds which are part of the “stain” class include, but are not limited to, atorvastatin, simvastatin, rosuvastatin, and pravastatin. Treatments usually administer from about 2 mg to 80 mg of a statin compound.

The inventors have found that HMG-CoA reductase inhibition leads to an increase in LDL receptor activity. Additionally, the inventors find that combining these two therapies leads to cooperative activity and favorable clinical treatment. Accordingly, the present invention is directed toward cholesterol-lowering compositions comprising statins and ETC-1002. These compositions lead to further reductions in total cholesterol, and specifically LDL-C, in patients.

The present application also discloses a method of lowering cholesterol using fixed dose combination of ETC-1002 and one or more statins. Based on observations in ongoing studies, combination therapy with ETC-1002 and a fixed, high dosage of one or more statins has comparable efficacy and safety to that of ETC-1002 combined with a fixed, low to medium dosage of one or more statins. Furthermore combination therapy with ETC-1002 and a fixed, high dosage of one or more statins is also significantly greater versus statin or ETC-1002 monotherapy (about 120 mg or about 180 mg daily) in patients with or without a history of statin-related muscle symptoms. The combination therapy shows a significantly greater efficacy and safety profile even in acute hypercholesterolemic patients.

In one aspect, the methods and compositions described in the present invention lower cholesterol in patients with persistently elevated LDL-C, despite receiving high dosages of statin therapy.

It is well known in the art that the chronic use of statin therapies in patients lead to an increase in HbA_{1C} level, a biomarker for diabetes. Such increase in HbA_{1C} level often times lead to a worsening of existing diabetic conditions, as well as increases the likelihood of developing new-onset diabetes in patients receiving high dosages of statin therapy.

Thus, the present application also discloses a method of improving HbA_{1C} level in patients with diabetes, or in patients who are at risk of developing new-onset diabetes.

### BRIEF DESCRIPTION OF THE FIGURES

These and other features, aspects, and advantages of the present invention will become better understood with regard to the following description, and accompanying drawings, where:

FIG. 1 depicts current understanding of the mechanism of action of bempedoic acid.

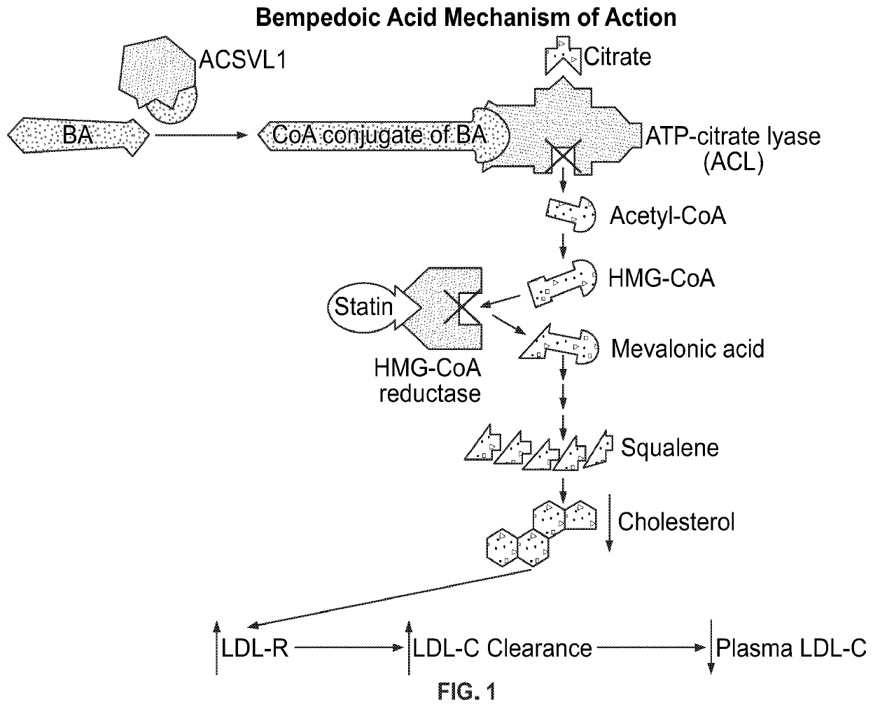

FIG. 2 depicts the observed change in HbA_{1C} levels in patients treated with bempedoic acid or placebo, at 12 and 52 weeks, where the patients have a medical history of diabetes and where the patients are receiving the maximal tolerated dose of statin (50% high intensity).

FIG. 3 depicts the change in fasting glucose levels over time for the same patients described in FIG. 2.

FIG. 4 depicts the observed change in HbA_{1C} levels in patients treated with bempedoic acid or placebo, at 12 and 24 weeks, where the patients have a medical history of diabetes, are statin-intolerant, and are being treated with low doses of statin (8% very low dose statin).

FIG. 5 depicts the change in fasting glucose levels over time for the same patients described in FIG. 4.

FIG. 6 depicts the observed change in HbA_{1C} levels in statin-intolerant patients treated with bempedoic acid or placebo for 12 weeks, where the patients are on a background level of ezetimibe, have a medical history of diabetes, and where 31% of the patients are on low or very low doses of statin.

FIG. 7 depicts the change in fasting glucose level over time for the same patients described in FIG. 6.

FIG. 8 depicts fasting glucose levels in patients with a medical history of diabetes, where the patients are on MTD high intensity statin (38.6%) or no statin (28%), and wherein the patients are treated for 12 weeks with (1) bempedoic acid+ezetimibe (top line; N=48; (2) bempedoic acid alone (middle line; N=57); (3) ezetimibe alone (bottom line; N=57); or (4) placebo (middle line; N=24).

### DETAILED DESCRIPTION

### Advantages and Utility

Briefly, and as described in more detail below, described herein are compositions, methods of making said compositions and methods for treating diabetes or reducing the risk of diabetes using any one of ETC-1002, ezetimibe, and any one of statins, or a combination of any one of ETC-1002, ezetimibe and a statin. The advantages for this approach are numerous and include, but are not limited to, increased reduction of cholesterol and low density lipoprotein levels in patients treated with the fixed-dose combinations of ETC-1002, ezetimibe and any one or more of statins as compared to the levels observed when patients are treated with ETC-1002 or ezetimibe or statins alone. While statins are the cornerstone of prevention and treatment of cardiovascular disease, they can produce unwanted side effects in many patients. Such side effects include, but are not limited to, increased concentrations of liver enzymes, muscle problems, an increased risk of diabetes, and worsening of existing diabetes. Statin-associated muscle symptoms are also an important clinical problem because statin discontinuation in hypercholesterolemic patients increases cardiovascular risk. Hence, there is a significant need for statin therapies for patients that exhibit muscle-related statin intolerance.

### Definitions

Terms used in the claims and specification are defined as set forth below unless otherwise specified.

Terms used in the claims and specification are defined as set forth below unless otherwise specified. Further, if any term or symbol used herein is not defined as set forth below, it shall have its ordinary meaning in the art.

As used herein and in the appended claims, singular articles such as “a,” “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, including the upper and lower bounds of the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

Generally, reference to a certain element such as hydrogen or H is meant to include all isotopes of that element. For example, if an R group is defined to include hydrogen or H, it also includes deuterium and tritium. Compounds comprising radioisotopes such as tritium, C¹⁴, P³² and S³⁵ are thus within the scope of the present technology. Procedures for inserting such labels into the compounds of the present technology will be readily apparent to those skilled in the art based on the disclosure herein.

The term “ameliorating” refers to any therapeutically beneficial result in the treatment of a disease state, e.g., an inflammatory disease state, including lessening in the severity or progression, remission, or cure thereof. In some embodiments, “ameliorating” includes prophylaxis of a disease state.

The term “diabetes” refers to diabetes mellitus, including Type I diabetes, Type 2 diabetes, and pre-diabetic conditions.

The term “cardiovascular diseases” refers to diseases of the heart and circulatory system. These diseases are often associated with dyslipoproteinemias and/or dyslipidemias. Cardiovascular diseases which the compositions of the present invention are useful for preventing or treating include but are not limited to arteriosclerosis; atherosclerosis; stroke; ischemic; endothelium dysfunctions, particularly those dysfunctions affecting blood vessel elasticity; peripheral vascular disease; coronary heart disease; myocardial infarction; cerebral infarction and restenosis.

The term “dyslipidemias” refers to disorders that lead to or are manifested by aberrant levels of circulating lipids. To the extent that levels of lipids in the blood are too high, the compositions of the invention are administered to a patient to restore normal levels. Normal levels of lipids are reported in medical treatises known to those of skill in the art. For example, recommended blood levels of LDL, HDL, free triglycerides and other parameters relating to lipid metabolism can be found at the web sites of the American Heart Association and that of the National Cholesterol Education Program of the National Heart, Lung and Blood Institute. Currently, the recommended level of HDL cholesterol in the blood is above 35 mg/dL; the recommended level of LDL cholesterol in the blood is below 130 mg/dL; the recommended LDL:HDL cholesterol ratio in the blood is below 5:1, ideally 3.5:1; and the recommended level of free triglycerides in the blood is less than 200 mg/dL.

The term “metabolic syndrome” refers to a cluster of conditions—increased blood pressure, high blood sugar, excess body fat around the waist, and abnormal cholesterol or triglyceride levels—that occur together, increasing your risk of heart disease, stroke and diabetes. These conditions are the co-occurrence of several known cardiovascular risk factors, including insulin resistance, obesity, atherogenic dyslipidemia and hypertension.

The term “nonalcoholic fatty liver disease (NAFLD)” refers to a condition in which excess fat is stored in your liver. This buildup of fat is not caused by heavy alcohol use. Nonalcoholic fatty liver disease (NAFLD) is characterized or diagnosed by the presence of fat in the liver (hepatic steatosis) either on imaging or on liver histology after the exclusion of secondary causes of fat accumulation in the liver (e.g., significant alcohol consumption, certain medications, and other medical conditions). NAFLD is further categorized histologically into nonalcoholic fatty liver (NAFL) and nonalcoholic steatohepatitis (NASH).

The term “simple fatty liver or nonalcoholic fatty liver (NAFL)” refers to a form of NAFLD in which you have fat in your liver but little or no inflammation or liver cell damage. NAFL is characterized with hepatic steatosis with no evidence of hepatocellular injury in the form of hepatocyte ballooning.

The term “nonalcoholic steatohepatitis (NASH)” refers to a form of NAFLD in which you have hepatitis—inflammation of the liver—and liver cell damage, in addition to fat in your liver. Inflammation and liver cell damage can cause fibrosis, or scarring, of the liver. NASH is characterized with the presence of hepatic steatosis and inflammation with hepatocyte injury (ballooning) with or without fibrosis.

The term “statin intolerant” refers to the occurrence of adverse symptoms perceived by the patient to be unacceptable (e.g., muscle-related symptoms), and/or laboratory abnormalities suggesting undue risk (e.g., serum liver enzyme activity), which are attributed to statin therapy and lead to its discontinuation.

The term “subject” refers to any mammal including humans, and so includes mammals such as those animals of veterinary and research interest that are including, but not limited to: simians, cattle, horses, dogs, cats, and rodents. The term “subject” is interchangeable with the term “patient.”

The term “mammal” as used herein includes both humans and non-human mammals, e.g., non-human primates, canines, felines, murines, bovines, equines, and porcines.

The term “administering” or “administration” of a drug and/or therapy to a subject (and grammatical equivalents of this phrase) refers to both direct or indirect administration, which may be administration to a subject by a medical professional, self-administration, and/or indirect administration, which may be the act of prescribing or inducing one to prescribe a drug and/or therapy to a subject.

The term “treating” or “treatment of” a disorder or disease refers to taking steps to alleviate the symptoms of the disorder or disease, or otherwise obtain some beneficial or desired results for a subject, including clinical results. Any beneficial or desired clinical results may include, but are not limited to, alleviation or amelioration of one or more symptoms of cancer or conditional survival and reduction of tumor load or tumor volume; diminishment of the extent of the disease; delay or slowing of the tumor progression or disease progression; amelioration, palliation, or stabilization of the tumor and/or the disease state; or other beneficial results.

The term “in vitro” refers to processes that occur in a living cell growing separate from a living organism, e.g., growing in tissue culture.

The term “in vivo” refers to processes that occur in a living organism.

The term “mammal” as used herein includes both humans and non-humans and include but is not limited to humans, non-human primates, canines, felines, murines, bovines, equines, and porcines.

The term “sufficient amount” means an amount sufficient to produce a desired effect, e.g., an amount sufficient to modulate protein aggregation in a cell.

The term “therapeutically effective amount” is an amount that is effective to ameliorate a symptom of a disease. A therapeutically effective amount can, in some embodiments, be a “prophylactically effective amount” as prophylaxis can be considered therapy.

The compounds of the present technology can exist as solvates, especially hydrates. Hydrates may form during manufacture of the compounds or compositions comprising the compounds, or hydrates may form over time due to the hygroscopic nature of the compounds. Compounds of the present technology can exist as organic solvates as well, including DMF, ether, and alcohol solvates among others. The identification and preparation of any particular solvate is within the skill of the ordinary artisan of synthetic organic or medicinal chemistry.

“Subject” refers to a mammalian organism treated using a compound of the present invention. The “subject” can be a human or non-human mammalian organism.

“Tautomer” refer to alternate forms of a compound that differ in the position of a proton, such as enol-keto and imine-enamine tautomers, or the tautomeric forms of heteroaryl groups containing a ring atom attached to both a ring NH moiety and a ring=N moiety such as pyrazoles, imidazoles, benzimidazoles, triazoles, and tetrazoles.

“Treating” or “treatment” of a disease or disorder in a subject refers to 1) preventing the disease or disorder from occurring in a subject that is predisposed or does not yet display symptoms of the disease or disorder; 2) inhibiting the disease or disorder or arresting its development; or 3) ameliorating or alleviating the cause of the regression of the disease or disorder.

As used herein, the terms “prevent,” “preventing,” “prevention,” “prophylactic treatment” and the like refer to reducing the probability of developing a disease, disorder, or condition in a subject, who does not have, but is at risk of or susceptible to developing a disease, disorder, or condition. Thus, in some embodiments, an agent can be administered prophylactically to prevent the onset of a disease, disorder, or condition, or to prevent the recurrence of a disease, disorder, or condition.

For the purposes of this specification and appended claims, the term “about,” when referring to a value can be meant to encompass variations of, in some aspects, ±100% in some aspects ±50%, in some aspects ±20%, in some aspects ±10%, in some aspects ±5%, in some aspects ±1%, in some aspects ±0.5%, and in some aspects ±0.1% from the specified amount, as such variations are appropriate to perform the disclosed methods or employ the disclosed compositions.

It is understood that in all substituted groups defined above, polymers arrived at by defining substituents with further substituents to themselves (e.g., substituted aryl having a substituted aryl group as a substituent which is itself substituted with a substituted aryl group, etc.) are not intended for inclusion herein. In such cases, the maximum number of such substituents is three. That is to say that each of the above definitions is constrained by a limitation that each functional group is substituted (at from one to three positions) and that any and all of those substituent groups may be substituted one more time (at from one to three positions).

It is understood that the above definitions are not intended to include impermissible substitution patterns (e.g., methyl substituted with 3 fluoro groups). Such impermissible substitution patterns are well known to the skilled artisan.

Throughout this application, the text refers to various embodiments of the present compounds, compositions, and methods. The various embodiments described are meant to provide a variety of illustrative examples and should not be construed as descriptions of alternative species. Rather, it should be noted that the descriptions of various embodiments provided herein may be of overlapping scope. The embodiments discussed herein are merely

### List of Abbreviations and Definitions of Terms

The following abbreviations and specialist terms are used in this study protocol.

TABLE 1
Abbreviations and Specialist Terms

| Abbreviation or | |
|---|---|
| Specialist Term | Explanation |
| ACL | Adenosine triphosphate-citrate lyase |
| ACS | Acyl-CoA synthetase |
| ADR | Adverse drug reaction |
| AE | Adverse event |
| ALB | Albumin |
| ALK-P | Alkaline phosphatase |
| ALT | Alanine aminotransferase |
| ANCOVA | Analysis of Covariance |
| apoA1 | Apolipoprotein A1 |
| apoB | Apolipoprotein B |
| aPTT | Activated partial thromboplastin time |
| ASCVD | Atherosclerotic cardiovascular disease |
| AST | Aspartate aminotransferase |
| ATP | Adenosine triphosphate |
| AUC | Area under the concentration-time curve |
| AUC_{0 24} | Area under the curve during 24 hours |
| AUCₗₐₛₜ | Area under the plasma concentration-time profile from |
| | time zero to the time of the last quantifiable |
| | concentration |
| BA | Bempedoic Acid |
| BLQ | Below the limit of quantification |
| BMI | Body mass index |
| BP | Blood pressure |
| BUN | Blood urea nitrogen |
| C₂₄ | Concentration in sample collected 24 hours |
| | post-dose, or prior to the next dose |
| Ca | Calcium |
| C_{avg} | Average plasma concentration over the dosing interval |
| CFR | Code of Federal Regulations |
| CHD | Coronary heart disease |
| CI | Confidence interval |
| CK | Creatine kinase |
| Cl | Chloride |
| CL/F | Apparent oral clearance |
| Cₘₐₓ | Observed maximum plasma concentration |
| Cₘᵢₙ | Minimum plasma concentration |
| CMV | Cytomegalovirus |
| CoA | Acetyl-coenzyme A |
| CO₂ | Carbon dioxide |
| CrCL | Creatinine clearance |
| CRF | Case report form |
| CT | Computed tomography |
| CYP | Cytochrome P450 |
| DBP | Diastolic blood pressure |
| ECG | Electrocardiogram |
| eCRF | Electronic case report form |
| EMA | European Medicines Agency |
| eGFR | Estimated glomerular filtration rate |
| EOS | End of study |
| ETC-1002 | Bempedoic Acid |
| EZE | Ezetimibe |
| FAS | Full analysis set |
| FDA | U.S. Food and Drug Administration |
| FDC | Fixed-Dose Combination |
| FPFV | First patient first visit |
| FSH | Follicle-stimulating hormone |
| GCP | Good Clinical Practice |
| GI | Gastrointestinal |
| HbA_{1C} | Glycosylated hemoglobin, Type A_{1C} |
| HBsAg | Hepatitis B surface antigen |
| Hct | Hematocrit |
| HCV | Hepatitis C virus |
| HDL-C | High-density lipoprotein cholesterol |
| HeFH | Heterozygous familial hypercholesterolemia |
| Hgb | Hemoglobin |
| HIV | Human immunodeficiency virus |
| HMG-CoA | 3-hydroxy-3-methylglutaryl coenzyme A |
| HR | Heart rate |
| hs-CRP | High-sensitivity C-reactive protein |
| IB | Investigator’s Brochure |
| ICD | Informed Consent Document |
| ICH | International Conference on Harmonisation |
| IEC | Independent Ethics Committee |
| IMP | Investigational Medicinal Product |
| IND | Investigational New Drug Application |
| INR | International normalized ratio |
| IRB | Institutional Review Board |
| ITT | Intention to Treat |
| IUD | Intrauterine device |
| IWRS | Interactive web response system |
| IVRS | interactive voice response system |
| K | Potassium |
| LDH | Lactate dehydrogenase |
| LDL-C | Low-density lipoprotein cholesterol |
| LDLR | LDL receptor |
| LFT | Liver function test |
| LOCF | Last observation carried forward |
| LPLV | Last patient last visit |
| LSM | Least squares means |
| MCH | Mean corpuscular hemoglobin |
| MCHC | Mean corpuscular hemoglobin concentration |
| MCV | Mean corpuscular volume |
| MDRD | Modification of diet in renal disease |
| MedDRA | Medical Dictionary for Regulatory Activities |
| MED | ID Medication identification |
| mITT | Modified intent-to-treat |
| Na | Sodium |
| NLA | National Lipid Association |
| NOAEL | No-observed-adverse-effect level |
| non-HDL-C | Non-high-density lipoprotein cholesterol |
| OLA | Open-label atorvastatin |
| PBO | Placebo |
| PCSK9 | Proprotein convertase subtilisin/kexin type 9 |
| PD | Pharmacodynamic |
| PE | Physical exam |
| PK | Pharmacokinetic(s) |
| PPAS | Per Protocol Analysis Set |
| PT | Prothrombin time |
| QD | Once daily |
| RBC | Red blood cell |
| SAE | Serious adverse event |
| SAP | Statistical Analysis Plan |
| SBP | Systolic blood pressure |
| SE | Standard error |
| SP | Safety population |
| t_{1/2} | Terminal elimination half-live |
| T2B | Type 2 diabetes |
| T2DM | Type 2 diabetes mellitus |
| TB | Total bilirubin |
| TC | Total cholesterol |
| TEAE | Treatment-emergent adverse event |
| TG | Triglycerides |
| tₘₐₓ | Time of observed maximum plasma concentration |
| TSH | Thyroid-stimulating hormone |
| TQT | Thorough QT/QTc |
| ULN | Upper limit of normal |
| USA | United States of America |
| WBC | White blood cell |

Therapy

Disclosed herein is a method comprising administrating a fixed-dose combination of any one of a fixed dose of ETC-1002 or an analog thereof, a fixed dose of ezetimibe or an analog thereof, and a fixed dose of one or more statins or an analog thereof to a subject in need thereof, optionally wherein ETC-1002 is administered at a fixed dose of about 180 mg or at a fixed dose of about 120 mg, ezetimibe is administered at a fixed dose of about 10 mg, and each one of the one or more statins is administered at a fixed dose from about 2 to about 80 mg.

In some aspects, the method decreases the level of low density lipoprotein cholesterol (LDL-C) in the subject below that of a control subject receiving a placebo.

In some aspects, the method comprises of administering a combination of any one of a fixed dose of ETC-1002 of about 120 mg, a fixed dose of ETC-1002 of about 180 mg, a fixed dose of ezetimibe of about 10 mg, or a fixed dose of each one of one or more statins of between about 2 to about 80 mg, and optionally wherein the method treats or reduces the risk of diabetes in the subject.

In some aspects, ETC-1002 is administered at a fixed dose of about 120 mg or at a fixed dose of about 180 mg, ezetimibe is administered at a fixed dose of about 10 mg, and statin is administered at a fixed dose of between 5-80 mg.

In some aspects, the subject has hypercholesterolemia, and wherein the method further comprises treating hypercholesterolemia.

In some aspects, the method treats or reduces the risk of cardiovascular disease in the subject.

In some aspects, the method treats or reduces diabetes in the subject.

In some aspects, the method treats or reduces the likelihood of developing new onset diabetes in the subject.

In some aspects, the method reduces HbA_{1C} level in patients receiving chronic statin therapy.

In some aspects, the method reduces HbA_{1C} level in patients with existing diabetic conditions.

In some aspects, the method reduces HbA_{1C} level in patients with Type 2 diabetes.

In some aspects, the method decreases the level of cholesterol in the subject below that of a control subject receiving a placebo.

In some aspects, the method decreases the level of C-reactive protein (hsCRP) in the subject below that of a control subject receiving a placebo.

In some aspects, the method decreases the level of apolipoprotein B (ApoB) in the subject below that of a control subject receiving a placebo.

In some aspects, the method decreases the level of non-high density lipoprotein-cholesterol in the subject below that of a control subject receiving a placebo.

In some aspects, the method decreases the level of triglycerides in the subject below that of a control subject receiving a placebo.

In some aspects, the method decreases the LDL particle number in the subject below that of a control subject receiving a placebo.

In some aspects, LDL-C level is decreased in the subject by at least 30, 35, 40, 43, 45, 48, or 50% relative to baseline.

In some aspects, non HDL-C level is decreased in the subject by at least 30, 35, 37, 40, 42, or 45% relative to baseline.

In some aspects, hsCRP level is decreased in the subject by at least 20, 25, 26, 30, 35, 38, or 40% relative to baseline.

In some aspects, the method improves glycemic control in the subject.

In some aspects, ETC-1002, ezetimibe, and statin are each administered orally.

In some aspects, ETC-1002, ezetimibe, and statin are each administered at least once daily.

In some aspects, ETC-1002, ezetimibe, and statin are each administered at least once daily for at least 1, 2, 3, 4, 5, 6, 7, 8, 9, 10, 11, or 12 week(s).

In some aspects, the subject has dyslipidemia.

In some aspects, the subject has hypercholesterolemia.

In some aspects, the subject is obese, optionally wherein the BMI of the subject is 18-45 kg/m².

In some aspects, the subject is statin tolerant.

In some aspects, the subject is statin intolerant.

In some aspects, the subject is unable to tolerate at least two statins including one statin at the lowest FDA approved dose due to muscle-related symptoms such as pain, aches, weakness, or cramping that began or increased during statin therapy and resolved when statin therapy was discontinued.

In some aspects, the subject has a baseline LDL-C level of 130-220 mg/dL.

In some aspects, the subject has a baseline triglycerides level of less than or equal to 400 mg/dL.

In some aspects, ETC-1002, ezetimibe, and statins are administered simultaneously.

In some aspects, ETC-1002, ezetimibe, and statins are administered separately.

Also disclosed herein is a pharmaceutical composition comprising ETC-1002, ezetimibe, and statins, optionally wherein ETC-1002 is present at a fixed dose of 120 mg or 180 mg, ezetimibe is present at a fixed dose of 10 mg, and statins are present at a fixed dose of between 5-80 mg.

In some aspects, the composition further comprises a pharmaceutically acceptable vehicle.

In some aspects, the composition is formulated for oral delivery.

In some aspects, the composition is formulated for administration once daily.

In some aspects, ETC-1002 is administered in an amount between 5 and 500 mg. In another aspect, ETC-1002 is administered in an amount between 10 and 450 mg. In another aspect, ETC-1002 is administered in an amount between 15 and 400 mg. In another aspect, ETC-1002 is administered in an amount between 20 and 350 mg. In another aspect, ETC-1002 is administered in an amount between 25 and 325 mg. In another aspect, ETC-1002 is administered in an amount between 30 and 300 mg. In another aspect, ETC-1002 is administered in an amount between 35 and 275 mg. In another aspect, ETC-1002 is administered in an amount between 40 and 250 mg. In another aspect, ETC-1002 is administered in an amount between 45 and 225 mg. In another aspect, ETC-1002 is administered in an amount between 50 and 200 mg.

In some aspects, the present disclosure provides for the administration of ETC-1002, wherein the dosage is 40 mg/day, 50 mg/day, 60 mg/day, 70 mg/day, 80 mg/day, 90 mg/day, 100 mg/day, 110 mg/day, 120 mg/day, 130 mg/day, 140 mg/day, 150 mg/day, 160 mg/day, 170 mg/day, 180 mg/day, 190 mg/day, 200 mg/day, 210 mg/day, 220 mg/day, 230 mg/day, 240 mg/day, or 250 mg/day.

In some aspects, the present disclosure provides for the administration of ETC-1002, wherein the dosage is 45-55 mg/day, 55-65 mg/day, 65-75 mg/day, 75-85 mg/day, 85-95 mg/day, 95-105 mg/day, 105-115 mg/day, 115-125 mg/day, 125-135 mg/day, 135-145 mg/day, 145-155 mg/day, 155-165 mg/day, 165-175 mg/day, 175-185 mg/day, 185-195 mg/day, 195-205 mg/day, 205-215 mg/day, 215-225 mg/day, 225-235 mg/day, 235-245 mg/day, or 245-255 mg/day.

In some embodiments, ezetimibe is administered in an amount between 1 and 50 mg; in another embodiment ezetimibe is administered in an amount between 5 and 25 mg; in another embodiment ezetimibe is administered in an amount between 5 and 15 mg; in another embodiment ezetimibe is administered in an amount between 1 and 10 mg; in another embodiment ezetimibe is administered in an amount between 10 and 20 mg; in another embodiment ezetimibe is administered in an amount between 8 and 12 mg; in another embodiment, ezetimibe is administered at a dose of 10 mg. Dosages are typically administered once a day. In some embodiments, dosages may be administered two, three, four, five times or more per day.

In some aspects, the subject has hypercholesterolemia, and wherein the method further comprises treating hypercholesterolemia.

In some aspects, the method treats or reduces the risk of cardiovascular disease in the subject.

In some aspects, the method decreases the level of cholesterol in the subject below that of a control subject receiving a placebo, a fixed dose of about 120 mg of ETC-1002, a fixed dose of about 180 mg dose of ETC-1002, or a fixed dose from about 2 to about 80 mg dose of each one of the one or more statins.

In some aspects, the method dose dependently reduces apolipoprotein B by about 10% to about 17% or more, non-high-density lipoprotein cholesterol by about 10% to about 17% or more, total cholesterol by about 10% to about 15% or more, and LDL particle number by about 10% to about 21% or more.

In some aspects, LDL-C is decreased in the subject up to about 24% or more relative to baseline. In some aspects, non HDL-C is decreased in the subject by at least about 30, about 35, about 37, about 40, about 42, or about 45% or more relative to baseline. In some aspects, hsCRP is decreased in the subject by at least about 20, about 25, about 26, about 30, about 35, about 38, or about 40% or more relative to baseline.

In some aspects, non-HDL-C is decreased in the subject by at least about 30, about 35, about 40, about 43, about 45, about 48, or about 50% or more relative to baseline. In other aspects, HDL-C is decreased in the subject relative to baseline.

In some aspects, HbA_{1C} level is decreased in the subject by at least about 0.1%, or 0.2%, or 0.3%, or 0.4%, or 0.5%, or 0.6% or 0.7%, or 0.8%, or 0.9%, or 1.0%, or 1.5%, or 1.7%, or 1.9%, or 2.0%, or 2.5%, or 3.0%, or 3.5%, or 4.0% as compared to subject not receiving the therapy or receiving the placebo.

In some aspects, the likelihood of new-onset diabetes is decreased in the subject by about 1%, or about 2%, or about 3%, or about 4%, or about 5%, or about 10%, or about 15%, or about 20%, or about 25%, or about 30%, or about 40%, or about 50%, or about 60% as compared to subject not receiving the therapy or receiving the placebo.

In some aspects, ETC-1002, ezetimibe, and statins are each administered orally.

In some aspects, ETC-1002, ezetimibe, and one or more of statins are each administered at least once daily.

In some aspects, ETC-1002, ezetimibe, and one or more of statins are each administered at least once daily for at least about 1, about 2, about 3, about 4, about 5, about 6, about 7, about 8, about 9, about 10, about 11, or about 12 week(s). In some related aspects, the administration of one or more of ETC-1002, ezetimibe, and at least one statin occurs less than at least once daily, e.g., once every other day, or once a week.

In some aspects, the subject experiences an adverse event when on one or more statins at the lowest FDA approved dose, said adverse events being selected form the group consisting of muscle-related pain, aches, weakness, and cramping. The inventors have observed that such muscle-related adverse events that began or increased during statin therapy could be significantly lowered or even resolved when treatment with add on ETC-1002 therapy to statin therapy was employed.

In some aspects, the subject has a baseline LDL-C level of about 115-220 mg/dL.

In some aspects, the subject has a baseline triglycerides level of less than or equal to about 400 mg/dL.

In some aspects, administration of a combination of bempedoic acid and ezetimibe to patients with both type 2 diabetes and hypercholesterolemia resulted in one or more of the following: reduction in LDL-C levels by up to 40 percent compared to placebo; reduction in levels of high-sensitivity C-reactive protein (hsCRP), an important marker of inflammation associated with cardiovascular disease, by up to 25 percent (p<0.001); a mean difference in hemoglobin A1c (HbA1c) of 0.03 percent compared to placebo; no change in overall adverse events (AEs) comparable to placebo; no increase in muscle-related AEs, serious adverse events, discontinuations due to AEs or elevations in liver function tests (LFTs); lower LDL-C levels to <70 mg/dl; reduction of LDL-C levels by >50 percent.

Also disclosed herein is a method of treating cardiovascular disease or reducing the risk of cardiovascular disease in a subject, comprising administrating a fixed-dose combination of a fixed dose of ETC-1002 or an analog thereof, a fixed dose of ezetimibe or an analog thereof, and a fixed dose of one or more statins or an analog thereof to a subject in need thereof, optionally wherein ETC-1002 is administered at a fixed dose of about 120 mg or at a fixed dose of about 180 mg, ezetimibe is administered at a fixed dose of about 10 mg, and each one of the one or more statins is administered at a fixed dose of between about 2 to about 80 mg, optionally wherein the method decreases the level of low density lipoprotein cholesterol (LDL-C) in the subject below that of a control subject receiving a placebo.

In some aspects, the method decreases the level of apolipoprotein B (ApoB) in the subject below that of a control subject receiving a placebo, a fixed dose of about 120 mg of ETC-1002, a fixed dose of about 180 mg of ETC-1002, a fixed dose of about 10 mg of ezetimibe, or a fixed dose of about 2 to about 80 mg dose for each one of one or more statins.

In some aspects, the method decreases the level of apolipoprotein A1 (ApoA1) in the subject below that of a control subject receiving a placebo, a fixed dose of about 120 mg of ETC-1002, a fixed dose of about 180 mg of ETC-1002, a fixed dose of about 10 mg of ezetimibe, or a fixed dose of about 2 to about 80 mg dose for each one of one or more statins.

In some aspects, the method does not change the level of ApoA1 in the subject compared to that of a control subject receiving a placebo, a fixed dose of about 120 mg of ETC-1002, a fixed dose of about 180 mg of ETC-1002, a fixed dose of about 10 mg of ezetimibe, or a fixed dose of about 2 to about 80 mg dose for each one of one or more statins.

In some aspects, the method decreases the ratio of ApoB to ApoA1 in the subject above that of a control subject receiving a placebo, a fixed dose of about 120 mg of ETC-1002, a fixed dose of about 180 mg of ETC-1002, a fixed dose of about 10 mg of ezetimibe, or a fixed dose of about 2 to about 80 mg dose for each one of one or more statins.

In some aspects, the method decreases the number of drug-related AEs by at least about 25%, by about 35%, by about 45% or by about 50% or more.

In some aspects, the method decreases the number of muscle-related AEs by at least about 50%, by about 65%, by about 75% or by about 85% or more.

In some aspects, the method disclosed herein significantly reduce the risk of a cardiovascular event in a subject. In some aspects this risk is reduced by up to about 35% or more.

In some aspects, the methods herein provide for treating cardiovascular disease and/or reducing the risk of cardiovascular disease in a subject comprising administering an amount of a composition comprising ETC-1002 which is rapidly absorbed having a Tₘₐₓ at less than about 4 hours.

In some aspects, the methods herein provide for treating cardiovascular disease and/or reducing the risk of cardiovascular disease in a subject comprising administering an amount of a composition comprising ETC-1002 which does not prolong QTc or QT/QTc (TQT study). In one aspect, the add-on ETC-1002 therapy does not affect subject heart rate and PR and QRS intervals.

In some aspects, the methods herein provide for treating cardiovascular disease and/or reducing the risk of cardiovascular disease in a subject comprising administering an amount of a composition comprising ETC-1002 which systemic exposure, AUC_{tau,ss}, occurs with t_{1/2} approximately about 15 to about 27 hours.

In some aspects, the methods herein provide for treating cardiovascular disease and/or reducing the risk of cardiovascular disease in a subject comprising administering an amount of a composition comprising ETC-1002 as add-on therapy to statin therapy which provides exposure measures AUC and/or Cₘₐₓ indicating that the 2 regimens have no appreciable drug interaction. In one embodiment, neither one or more statin(s) nor ETC-1002 exposure measures are outside safe values as established by confidence intervals.

In one aspect, the composition includes one or more statins as defined by the fixed dosages of atorvastatin (10 mg or 20 mg), simvastatin (5 mg, 10 mg, or 20 mg), rosuvastatin (5 mg or 10 mg), and/or pravastatin (10 mg, 20 mg, or 40 mg). In another aspect, the method includes one or more statins as defined by the fixed dosages of atorvastatin (10 mg or 20 mg), simvastatin (5 mg, 10 mg, or 20 mg), rosuvastatin (5 mg or 10 mg), and/or pravastatin (10 mg, 20 mg, or 40 mg). In yet another aspect, any combination of atorvastatin (10 mg or 20 mg), simvastatin (5 mg, 10 mg, or 20 mg), rosuvastatin (5 mg or 10 mg), and/or pravastatin (10 mg, 20 mg, or 40 mg) may be used in any embodiment or aspect disclosed herein.

In one aspect, the composition includes one or more statins as defined by the fixed dosages of Table 2 below:

TABLE 2

| High Intensity | Moderate | Low Intensity |
|---|---|---|
| Statins | Intensity Statins | Statins |
| Atorvastatin 40-80 mg | Atorvastatin 10-20 mg | Simvastatin 10 mg |
| Rosuvastatin 20-40 mg | Rosuvastatin 5-10 mg | Pravastatin 10-20 mg |
| Simvastatin 80 mg^{‡} | Simvastatin 20-40 mg | Lovastatin 20 mg |
| | Pravastatin 40-80 mg | Fluvastatin 20-40 mg |
| | Lovastatin 40 mg | Pitavastatin 1 mg |
| | Fluvastatin XL 80 mg | |
| | Fluvastatin 40 mg | |
| | twice daily | |
| | Pitavastatin 2-4 mg | |

### Compounds

Combinations of one or more statins and ETC-1002 are described herein. In one aspect, one or more or all of the statins are natural products isolated from a natural source such as Penecillium and Aspergillus fungi. In another aspect, one or more or all of the statins are synthetic, meaning they are made by advancing petrochemical starting material via organic synthesis to the desired statin compound.

Formula I below shows ETC-1002 and analogs of ETC-1002.

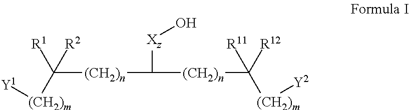

wherein (a) each occurrence of m is independently an integer ranging from 0 to 5; (b) each occurrence of n is independently an integer ranging from 3 to 7; (c) X is (CH₂), or Ph, wherein z is an integer from 0 to 4 and Ph is a 1,2-, 1,3-, or 1,4 substituted phenyl group; (d) each occurrence of R¹, R², R¹¹, and R¹² is independently H, (C₁-C₆)alkyl, (C₂-C₆)alkenyl, (C₂-C₆)alkynyl, phenyl, or benzyl, wherein R¹, R², R¹¹, and R¹² are not each simultaneously H; and (e) each occurrence of Y¹ and Y² is independently (C₁-C₆)alkyl, OH, COOH, COOR³, SO.₃H,

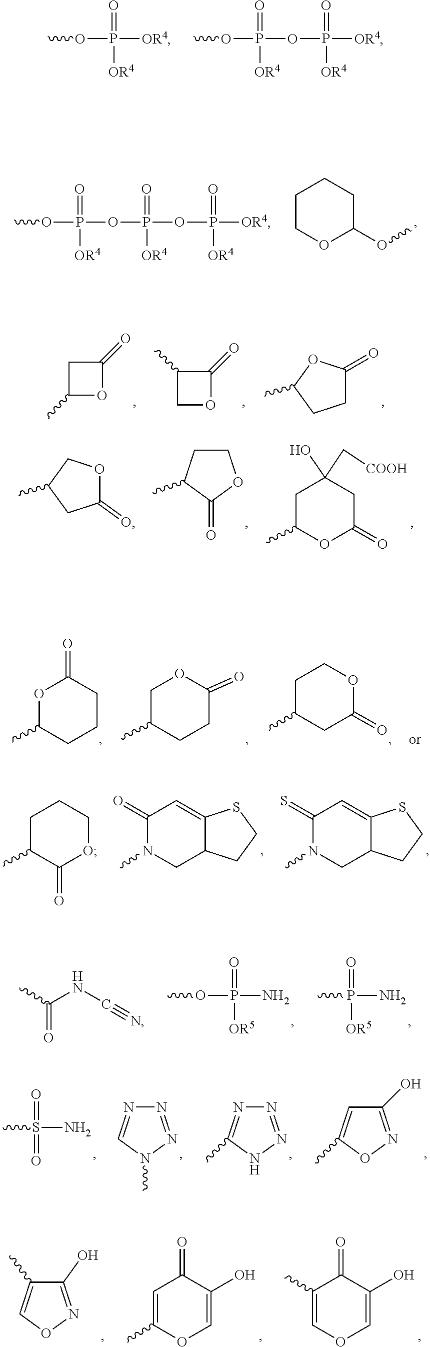

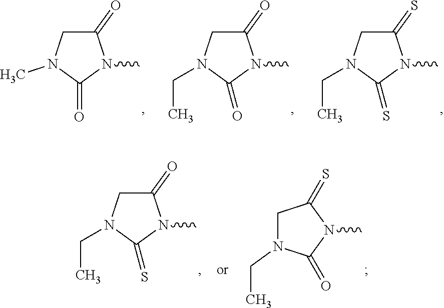

wherein: (i) Y¹ and Y² are not each simultaneously (C₁-C₆)alkyl; (ii) R³ is (C₁-C₆)alkyl, (C₂-C₆)alkenyl, (C₂-C₆)alkynyl, phenyl, or benzyl and is unsubstituted or substituted with one or more halo, OH, (C₁-C₆)alkoxy, or phenyl groups, (iii) each occurrence of R⁴ is independently H, (C₁-C₆)alkyl, (C₂-C₆)alkenyl, or (C₂-C₆)alkynyl and is unsubstituted or substituted with one or two halo, OH, C₁-C₆, alkoxy, or phenyl groups; and (iv) each occurrence of R⁵ is independently H, (C₁-C₆)alkyl, (C₂-C₆)alkenyl, or (C₂-C₆)alkynyl.

Structure of ETC-1002:

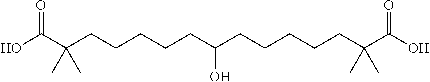 [SMILES: CC(C)(CCCCCC(O)CCCCCC(C)(C)C(=O)O)C(=O)O]

ETC-1002 can be referred to as 8-hydroxy-2,2,14,14 tetramethylpentadecanedioic acid.

Statin compounds inhibit HMGR enzymatic activity in the liver. In terms of structure, all statin compounds possess a dihydroxyheptanoic acid group or lactone thereof and a substituted ring system (shown below).

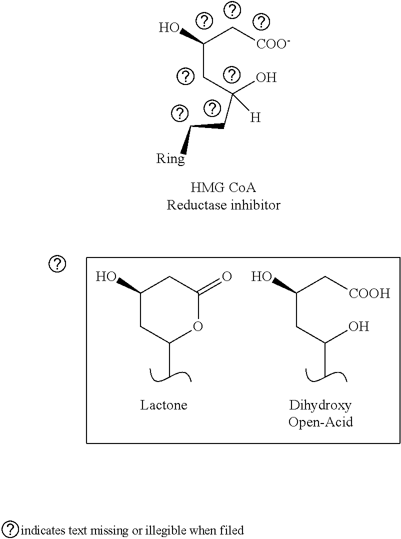

However, statins do differ with respect to the substituted ring structure. Some statins have a substituted decalin-ring structure while others have substituted aryl and heteroaryl ring systems. The structure of exemplary statin compounds is shown below, however, this list is in no way limiting.

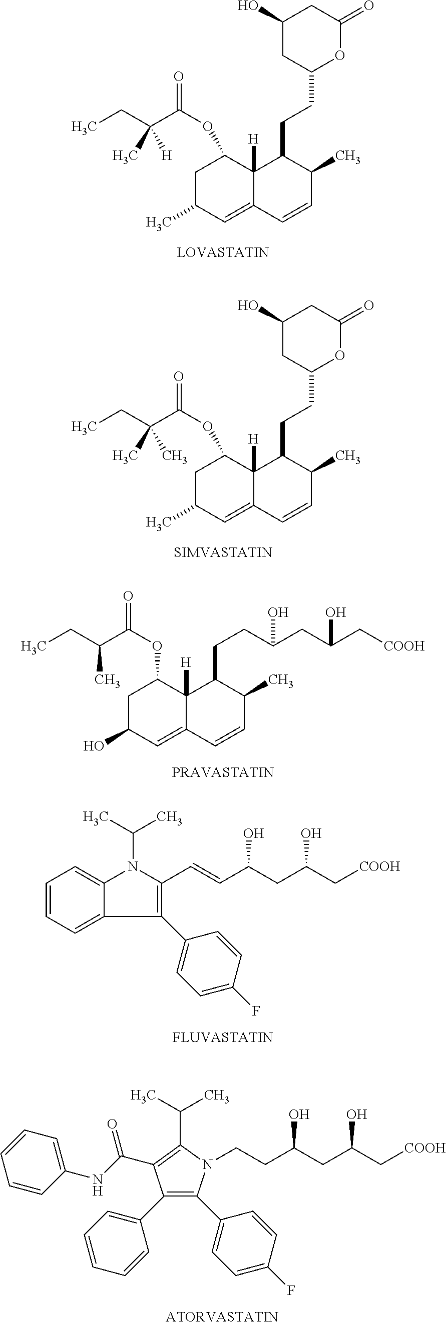

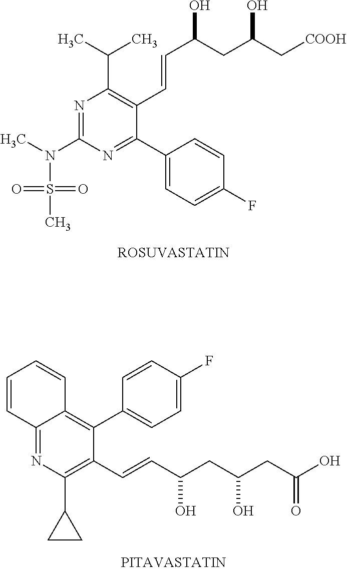 [SMILES: CC(C)c1nc(N(C)S(C)(=O)=O)nc(-c2ccc(F)cc2)c1/C=C/[C@@H](O)C[C@@H](O)CC(=O)O ; O=C(O)C[C@H](O)C[C@H](O)/C=C/c1c(C2CC2)nc2ccccc2c1-c1ccc(F)cc1]

It is acknowledged that any and all analogs of ETC-1002 according to Formula I can be used in any of the methods and/or compositions or formulations disclosed herein. It is further acknowledged that any and all analogs of statins according to the description above can be used in any of the methods and/or compositions or formulations disclosed herein Formula (II) below shows Ezetimibe and analogs of Ezetimibe

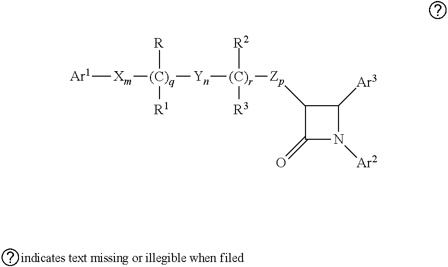

wherein in Formula (II) above or a salt thereof, wherein: Ar¹ and Ar² are independently selected from the group consisting of aryl and R⁴-substituted aryl; Ar³ is aryl or R⁵-substituted aryl; X, Y and Z are independently selected from the group consisting of —CH₂—, —CH(lower alkyl)- and —C(dilower alkyl)-; R and R² are independently selected from the group consisting of —OR⁶, —O(CO)R⁶, —O(CO)OR⁹ and —O(CO)NR⁶R⁷; R¹ and R³ are independently selected from the group consisting of hydrogen, lower alkyl and aryl; q is 0 or 1; r is 0 or 1; m, n and p are independently selected from 0, 1, 2, 3 or 4; provided that at least one of q and r is 1, and the sum of m, n, p, q and r is 1, 2, 3, 4, 5 or 6; and provided that when p is 0 and r is 1, the sum of m, q and n is 1, 2, 3, 4 or 5; R⁴ is 1-5 substituents independently selected from the group consisting of lower alkyl, —OR⁶, —O(CO)R⁶, —O(CO)OR⁹, —O(CH₂)₁₋₅ OR⁶, —O(CO)NR⁶R⁷, —NR⁶R⁷, —NR⁶ (CO)R⁷, —NR⁶ (CO)OR⁹, —NR⁶ (CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀ CONR⁶R⁷, -(lower alkylene)COOR⁶, —CH═CH—COOR⁶, —CF₃, —CN, —NO₂ and halogen; R⁵ is 1-5 substituents independently selected from the group consisting of —OR⁶, —O(CO)R⁶, —O(CO)OR⁹, —O(CH₂)₁₋₅ OR⁶, —O(CO)NR⁶R⁷, —NR⁶R⁷, —NR⁶ (CO)R⁷, —NR⁶ (CO)OR⁹, —NR⁶ (CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀ CONR⁶R⁷, -(lower alkylene)COOR⁶ and —CH═CH—COOR⁶; R⁶, R⁷ and R⁸ are independently selected from the group consisting of hydrogen, lower alkyl, aryl and aryl-substituted lower alkyl; and R⁹ is lower alkyl, aryl or aryl-substituted lower alkyl.

Ezetimibe can be referred to as 1-(4-fluorophenyl)-3(R)-[3(S)-(4-fluorophenyl)-3-hydroxypropyl)]-4(S)-[4-(phenylmethoxy)phenyl]-2-azetidinone; or (3R,4S)-1-(4-fluorophenyl)-3-[(3S)-3-(4-fluorophenyl)-3-hydroxypropyl]-4-(4-hydroxyphenyl)azetidin-2-one.

The structure of Ezetimibe is:

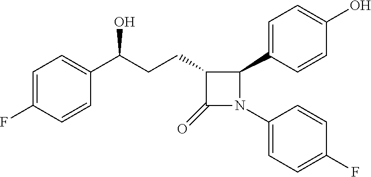 [SMILES: O=C1[C@H](CC[C@H](O)c2ccc(F)cc2)[C@@H](c2ccc(O)cc2)N1c1ccc(F)cc1]

It is acknowledged that any and all analogs of ETC-1002 according to Formula I can be used in any of the methods and/or compositions or formulations disclosed herein. It is further acknowledged that any and all analogs of ETC-1002 according to Formula II can be used in any of the methods and/or compositions or formulations disclosed herein.

### Synthesis of ETC-1002, Ezetimibe and Statins

ETC-1002 and the process of synthesis of ETC-1002 is disclosed in the issued U.S. Pat. No. 7,335,799. The details of this process can be found in the published U.S. patent publication No. US2005-0043278 A1, in paragraphs [0247]-[0343] of the specification, each of which is herein incorporated by reference.

Ezetimibe and the process of synthesis of Ezetimibe is disclosed in the issued U.S. Pat. No. 5,631,365. The details of this process can be found in the specification, beginning on page 4 right column, line 43 through page 11 right column, line 65, each of which is herein incorporated by reference.

The synthesis of statins is known in the art. In a strategic and general disclosure, the synthesis of statins is disclosed in WO2005047276A2 which is herein incorporated by reference. Any other synthetic modifications for statins (or analogs of ETC-1002 for that matter), which may include unique or alternative ring systems, are within the purview of the skilled artisan. For example, the skilled artisan will be able to use synthetic reference texts to incorporate unique or desired substituted-aryl, heteroaryl, and decalin ring systems into the final statin compound. Such references include, hut are not limited to: as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989), Organic Reactions, Volumes 1-40 (John Wiley, and Sons, 1991), March's Advanced Organic Chemistry, (John Wiley, and Sons, 5^{th} Edition, 2001), and Larock's Comprehensive Organic Transformations (VCH Publishers Inc., 1989), T. W. Greene and P. G. M. Wuts, Protecting Groups in Organic Synthesis, Third Edition, Wiley, New York, 1999.

### Methods of Use

The present invention provides methods for the treatment or prevention of a cardiovascular disease, comprising administering to a subject fixed doses of compounds or a composition comprising compounds of the invention and a pharmaceutically acceptable vehicle. As used herein, the term “cardiovascular diseases” refers to diseases of the heart and circulatory system. These diseases are often associated with dyslipoproteinemias and/or dyslipidemias. Cardiovascular diseases which the compositions of the present invention are useful for preventing or treating include but are not limited to arteriosclerosis; atherosclerosis; stroke; ischemia; endothelium dysfunctions, in particular those dysfunctions affecting blood vessel elasticity; peripheral vascular disease; coronary heart disease; myocardial infarction; cerebral infarction and restenosis.

The present invention provides methods for the treatment or prevention of a dyslipidemia comprising administering to a subject fixed doses of compounds or a composition comprising compounds of the invention and a pharmaceutically acceptable vehicle. As used herein, the term “dyslipidemias” refers to disorders that lead to or are manifested by aberrant levels of circulating lipids. To the extent that levels of lipids in the blood are too high, the compositions of the invention are administered to a patient to restore normal levels. Normal levels of lipids are reported in medical treatises known to those of skill in the art. For example, recommended blood levels of LDL, HDL, free triglycerides and others parameters relating to lipid metabolism can be found at the web site of the American Heart Association and that of the National Cholesterol Education Program of the National Heart, Lung and Blood Institute (http://www.americanheartorg/cholesterol-/about_level.html and http://www.nhlbi.nih.gov/health/public/heart/chol/hb-c_what.html, respectively). At the present time, the recommended level of HDL cholesterol in the blood is above about 35 mg/dL; the recommended level of LDL cholesterol in the blood is below about 130 mg/dL; the recommended LDL:HDL cholesterol ratio in the blood is below about 5:1, ideally about 3.5:1; and the recommended level of free triglycerides in the blood is less than about 200 mg/dL.

Dyslipidemias which the compositions of the present invention are useful for preventing or treating include but are not limited to hyperlipidemia and low blood levels of high density lipoprotein (HDL) cholesterol. In certain embodiments, the hyperlipidemia for prevention or treatment by the compounds of the present invention is familial hypercholesterolemia; familial combined hyperlipidemia; reduced or deficient lipoprotein lipase levels or activity, including reductions or deficiencies resulting from lipoprotein lipase mutations; hypertriglyceridemia; hypercholesterolemia; high blood levels of urea bodies (e.g. .beta.-OH butyric acid); high blood levels of Lp(a) cholesterol; high blood levels of low density lipoprotein (LDL) cholesterol; high blood levels of very low density lipoprotein (VLDL) cholesterol and high blood levels of non-esterified fatty acids.

The present invention provides methods for altering lipid metabolism in a patient, e.g., reducing LDL in the blood of a patient, increasing the ratio of HDL to LDL in the blood of a patient, and inhibiting saponified and/or non-saponified fatty acid synthesis, said methods comprising administering to the patient a compound or a composition comprising a compound of the invention in an amount effective alter lipid metabolism.

The present invention provides methods of reducing HbA_{1C} level in subjects who are receiving chronic statin therapy.

The present invention provides methods of reducing HbA_{1C} level in subjects who are diabetic.

The present invention further provides methods of reducing HbA_{1C} in subjects who are at risks of developing new-onset diabetes.

In some aspects, HbA_{1C} level is decreased in the subject by at least about 0.1%, or 0.2%, or 0.3%, or 0.4%, or 0.5%, or 0.6% or 0.7%, or 0.8%, or 0.9%, or 1.0%, or 1.5%, or 1.7%, or 1.9%, or 2.0%, or 2.5%, or 3.0%, or 3.5%, or 4.0% as compared to subject not receiving the therapy or receiving the placebo.

In some aspects, the likelihood of new-onset diabetes is decreased in the subject by about 1%, or about 2%, or about 3%, or about 4%, or about 5%, or about 10%, or about 15%, or about 20%, or about 25%, or about 30%, or about 40%, or about 50%, or about 60% as compared to subject not receiving the therapy or receiving the placebo.

### Pharmaceutical Compositions

Methods for treatment of cardiovascular diseases are also encompassed by the present invention. Said methods of the invention include administering a therapeutically effective amount of one or more statins, ETC-1002 and ezetimibe. The fixed dose combination of any one of one or more statins, ETC-1002 and ezetimibe can be formulated in pharmaceutical compositions. The fixed dose combination of ETC-1002 and ezetimibe can also be formulated in pharmaceutically compositions. These compositions comprise a pharmaceutically acceptable excipient, carrier, buffer, stabilizer or other materials well known to those skilled in the art. Such materials should be non-toxic and should not interfere with the efficacy of the active ingredient. The precise nature of the carrier or other material can depend on the route of administration, e.g. oral, intravenous, cutaneous or subcutaneous, nasal, intramuscular, intraperitoneal routes.

Pharmaceutical compositions for oral administration can be in tablet, capsule, pill, powder or liquid form. A tablet or pill can include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol can be included.

In one aspect, pharmaceutical compositions of the present invention are created from one or more of the compounds disclosed herein and are in the form of a pill.

In another aspect, herein provided is a method for lowering cholesterol or the associated markers disclosed herein (HDL-C, ApoA1, etc.) or for the treatment or prevention of a cardiovascular disease or dyslipoproteinemias and/or dyslipidemias, comprising administering to a subject a pharmaceutical composition in the form of a pill comprising ETC-1002 at a fixed dose of about 120 mg or about 180 mg, a fixed dose from about 2 to about 80 mg dose of each one of one or more statins, and a fixed dose of about 10 mg of ezetimibe.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives can be included, as required.

Whether it is a small molecule or other pharmaceutically useful compound according to the present invention that is to be given to an individual, administration is preferably in a “therapeutically effective amount” or “prophylactically effective amount” (as the case can be, although prophylaxis can be considered therapy), this being sufficient to show benefit to the individual. The actual amount administered, and rate and time-course of administration, will depend on the nature and severity of protein aggregation disease being treated. Prescription of treatment is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

A composition can be administered alone or in combination with other treatments, either simultaneously or sequentially dependent upon the condition to be treated.

In one aspect the present disclosure provides for a method of treating cardiovascular disease or reducing the risk of cardiovascular disease in a subject, comprising administrating a fixed dose of ETC-1002 or an analog thereof, a fixed dose of one or more statins or an analog thereof, and a fixed dose of ezetimibe to a subject in need thereof, optionally wherein ETC-1002 is administered at a fixed dose of about 120 mg or at a fixed dose of about 180 mg, each of the one more statins is administered at a fixed dose between about 2 about 80 mg, and ezetimibe is administered at a fixed dose of about 10 mg, and optionally wherein the method treats or reduces the risk of diabetes in the subject.

In one aspect the present disclosure provides for a method wherein the level of total cholesterol and non-HDL-C in the subject is below that of a control subject receiving placebo, a fixed dose of about 120 mg of ETC-1002, a fixed dose of about 180 mg of ETC-1002, a fixed dose of about 10 mg of ezetimibe, or a fixed dose of about 2 to about 80 mg dose for each one of one or more statins.

In one aspect the present disclosure provides for a method wherein the level of low density lipoprotein (LDL) in the subject is below that of a control subject receiving placebo, a fixed dose of about 120 mg of ETC-1002, a fixed dose of about 180 mg dose of ETC-1002, a fixed dose of about 10 mg of ezetimibe, or a fixed dose of about 2 to about 80 mg dose for each one of one or more statins.

In one aspect the present disclosure provides for a method wherein the number of LDL particles in the subject is below that of a control subject receiving placebo, a fixed dose of about 120 mg of ETC-1002, a fixed dose of about 180 mg of ETC-1002, a fixed dose of about 10 mg of ezetimibe, or a fixed dose of about 2 to about 80 mg dose for each one of one or more statins.

In one aspect the present disclosure provides for a method wherein the level of apolipoprotein B (ApoB) in the subject is less than that of a control subject receiving placebo, a fixed dose of about 120 mg of ETC-1002, a fixed dose of about 180 mg of ETC-1002, a fixed dose of about 10 mg of ezetimibe, or a fixed dose of about 2 to about 80 mg for each one of one or more statins.

In one aspect the present disclosure provides for a method wherein the level of apolipoprotein A-1 (ApoA1) in the subject is less than that of a control subject receiving placebo, a fixed dose of about 120 mg of ETC-1002, a fixed dose of about 180 mg of ETC-1002, a fixed dose of about 10 mg of ezetimibe, or a fixed dose of about 2 to about 80 mg for each one of one or more statin

In one aspect the present disclosure provides for a method wherein the ratio of apolipoprotein B (ApoB) to apolipoprotein A-1 (ApoA1) in the subject is below that of a control subject receiving placebo, a fixed dose of about 120 mg of ETC-1002, a fixed dose of about 180 mg of ETC-1002, a fixed dose of about 10 mg of ezetimibe, or a fixed dose of about 2 to about 80 mg for each one of one or more statins.

In one aspect the present disclosures provide for a method wherein the HbA_{1C} level in the subject is below that of a control subject receiving placebo, a fixed dose of about 120 mg of ETC-1002, a fixed dose of about 180 mg of ETC-1002, a fixed dose of about 10 mg of ezetimibe, or a fixed dose of about 2 to about 80 mg for each one of one or more statins.

In one aspect the present disclosure provides for a method wherein the subject has hypercholesterolemia.

In one aspect, the present disclosure provides for a method wherein the subject has diabetes.

In one aspect, the present disclosure provides for a method wherein the subject has Type 2 diabetes.

In one aspect, the present disclosure provides for a method wherein the subject has risk factors of developing new onset diabetes.

In one aspect the present disclosure provides for a method wherein the subject is human.

In one aspect the present disclosure provides for a therapeutic composition of comprising a therapeutic amount of a fixed dose of ETC-1002, ezetimibe, and a fixed dose for each one of one or more statins.

### Background on ETC-1002

### Mechanism of Action

ETC-1002 is a small molecule inhibitor of adenosine triphosphate (ATP)-citrate lyase (ACL), an enzyme upstream of hydroxymethyl glutaryl coenzyme A (HMG-CoA) reductase (molecular target of statins) in the cholesterol biosynthesis pathway. ETC-1002 can mediate competitive inhibition of ACL. Inhibition of ACL decreases cholesterol synthesis in liver leading to increased LDLR expression and LDL particle clearance from the blood. Therefore, inhibition of ACL by ETC-1002 is via the same pathway as HMG-CoA reductase inhibition by statins.

An important differentiating feature of ETC-1002 is that, unlike statins, it does not inhibit cholesterol synthesis in skeletal muscle. Therefore, ETC-1002 is not expected to cause the adverse effects associated with inhibition of the cholesterol biosynthesis pathway in skeletal muscle.

In the Examples described herein, unless otherwise stated, the daily dose of bempedoic acid (180 mg or 120 mg) is taken as an individual tablet or in combination with EZE in the fixed dose combination (FDC) tablet represents the dose that is being evaluated in Phase 3 monotherapy and combination therapy trials of bempedoic acid in subjects with hypercholesterolemia. The daily dose of EZE (10 mg) taken as an individual tablet or in combination with BA in the FDC tablets represents the recommended therapeutic dose for this drug. Additional details about these trials appear below.

### Subject Inclusion Criteria

The subject must be willing to provide written informed consent before any study specific procedures are performed.

The subject must be aged 18-75 years or be of legal age of majority based on regional law, whichever is older.

The subject must have a history of T2D for 6 months or greater; and must be currently taking stable diabetes medication for 3 months or greater with HbA1C between 7% and 10% at Visit S1.

The subject must have a fasting calculated LDL-C level >70 mg/dL at Visit S1.

The subject must have a fasting calculated LDL-C level between 100 and 220 mg/dL at Visit S3 after washout of all LMT.

The subject must clinically stable and suitable to undergo washout of all LDL-C-lowering drugs and nutritional supplements for 17 weeks (with potential for 1-week extension if repeat assessments described in the protocol are required) based on investigator assessment.

The subject may be male or female. Women must not be pregnant (or planning to become pregnant within 30 days after the last dose of IMP) or lactating and must be:

a. Naturally postmenopausal, defined as ≥1 year without menses and either:
i. ≥55 years old, or
ii. ≤55 years old with a follicle-stimulating hormone (FSH) level ≥40.0 IU/L;

b. Surgically sterile by hysterectomy, bilateral oophorectomy, and/or tubal ligation; or

c. Willing to use 1 acceptable method of birth control if of childbearing potential unless the subject agrees to follow the definition of true abstinence. The minimal requirement for use of acceptable contraception is from the time the informed consent form (ICF) is signed, during the study period, and for at least 30 days after the last dose of IMP. Acceptable methods of birth control include:
i. placement of an intrauterine device (IUD) with or without hormones,
ii. established use of oral, implanted, topical, or injectable, or hormonal method of contraception associated with inhibition of ovulation,
iii. barrier methods, including condom or occlusive cap with spermicidal foam or spermicidal jelly,
iv. vasectomized male partner who is the sole partner for the subject, or
v. true abstinence when this is in line with the preferred and usual lifestyle of the subject. Periodic abstinence (e.g., calendar, ovulation, symptothermal, post ovulation methods), declaration of abstinence for the duration of a trial, and withdrawal are not acceptable methods of contraception.

### Subject Exclusion Criteria

The subject has a body mass index (BMI) >40 kg/m².

The subject has a history of documented clinically significant cardiovascular disease including, but not limited to myocardial infarction, severe or unstable angina pectoris, coronary angioplasty, coronary artery bypass graft, stroke, transient ischemic attack, cerebrovascular event, symptomatic carotid artery disease, or symptomatic peripheral arterial disease, uncontrolled hypertension, defined as mean systolic blood pressure ≥160 mmHg and/or diastolic blood pressure ≥100 mmHg after sitting quietly for 5 minutes, an arrhythmia requiring medical intervention, abdominal or thoracic aortic aneurysm.

The subject has a history of type 1 diabetes.

The subject has a fasting TG level >400 mg/dL at Visit S3.

The subject has uncontrolled hypothyroidism, including a value for thyroid-stimulating hormones (TSH) >1.5 times the upper limit of normal.

The subject has liver disease or dysfunction, including positive serology for hepatitis B surface antigen (HBsAg) and/or hepatitis C virus antibodies (HCV-AB) at Week 1 (Visit S2/Day 7), or serum alanine aminotransferase (ALT) or aspartate aminotransferase (AST) value ≥2×ULN and/or serum total bilirubin (TB) value ≥2×ULN. If the serum TB value is ≥1.2×ULN, a reflex indirect (unconjugated) bilirubin will be obtained and, if consistent with Gilbert's disease or if the subject has a history of Gilbert's disease, the subject may be enrolled in the study.

The subject has renal dysfunction or glomerulonephritis, including an estimate glomerular filtration rate (eGFR)<30 mL/min/1.73 m2 (as determined by the central laboratory using the Modification of Diet in Renal Disease [MDRD] formula).

The subject has gastrointestinal conditions or has undergone procedures (including weight loss surgery; eg, Lap-Band, gastric bypass) that may affect drug absorption.

The subject has hematologic or coagulation disorders or a hemoglobin (Hgb) level <10.0 g/dL.

The subject had an active malignancy, including those requiring surgery, chemotherapy, and/or radiation, in the past 5 years. Nonmetastatic basal or squamous cell carcinoma of the skin and cervical carcinoma in situ are allowed.

The subject has an unexplained (i.e., not associated with recent trauma or physically strenuous activity) serum creatine kinase (CK) value >3×ULN at any time before randomization. Subjects with an explained elevation in serum CK must have single repeat serum CK value ≤3×ULN before randomization.

The subject has a history of drug or alcohol abuse within the last 2 years or reports current consumption of >14 alcoholic drinks/week, uses any illicit drugs, or has a history of amphetamine or derivatives abuse or cocaine abuse. Subjects who are using amphetamine derivatives prescribed by and who are under the care of a health care practitioner can be enrolled after evaluation by the investigator.

The subject has donated blood, undergone multiple blood draws in a clinical study, experienced major trauma, received a blood transfusion, or undergone surgery, with or without blood loss, within 30 days before randomization.

The subject has used any experimental or investigational drugs within 30 days before screening and throughout the trial.

The subject has previously participated in a clinical study of BA.

The subject has experienced history of intolerance to EZE.

The subject has used prohibited drugs and/or nutritional supplements within 5 weeks prior to Visit T1 (unless otherwise specified) or plans to use any of the prohibited drugs and/or nutritional supplements during the study, including but not limited to statins, fibrates (including fenofibrate), niacin and derivatives, bile acid sequestants, ezetimibe (study-provided is allowed), apheresis, mipomersen or lomitapide (6 months prior to Visit S1), proprotein convertase subtilisin/kexin type 9 (PCSK9) inhibitors (4 months prior to Visit S1, except PCSK9 small interfering RNA (siRNA), which are prohibited if used at any time in the past), cholesteryl ester transfer protein (CETP) inhibitors (12 months prior to Visit S1);], red yeast rice extract-containing products, omega 3 fatty acids and derivatives such as Lovaza® and over-the-counter (OTC) fish oil.

### Risk Benefit Summary

To date, the nonclinical and clinical data indicate that ETC-1002 has a favorable risk-benefit profile. The ability of ETC-1002 to achieve clinically meaningful LDL-C-lowering responses while demonstrating a favorable tolerability profile in a variety of patient populations supports continued development of ETC-1002, an oral ACL inhibitor.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

Patients with diabetes were identified primarily through medical history and/or baseline lab results. No entry restrictions were placed on these patients with the exception of having a HbA1c <10% during screening. The use of background concomitant medications related to diabetes treatment was captured at the study outset. Changes in all concomitant medications were tracked during the treatment phase. No restrictions were placed on changes in diabetes medications (stopping, starting, or changing dose) during the trial.

### Example 1: Bempedoic Acid Reduces Diabetes Risk in Patients Receiving High Intensity Statin Therapy

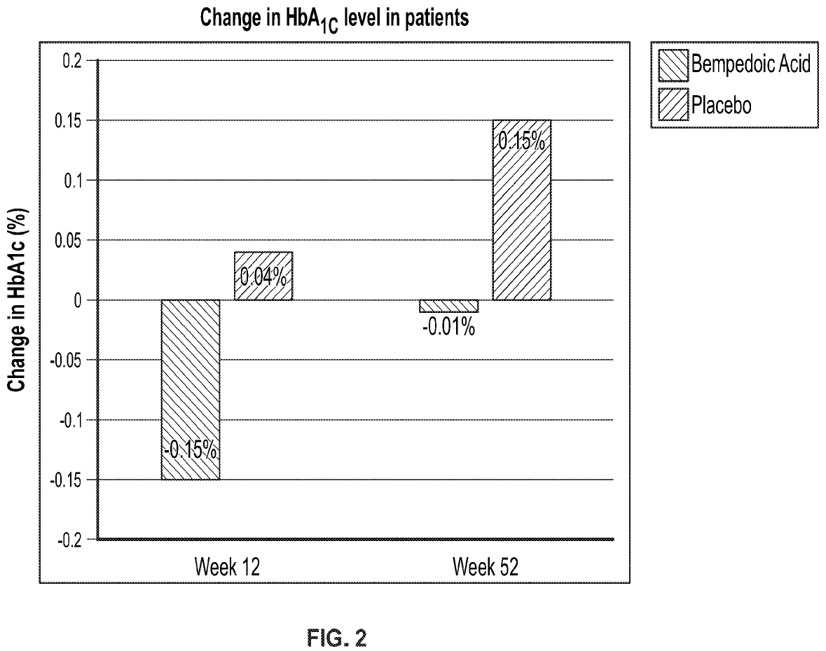

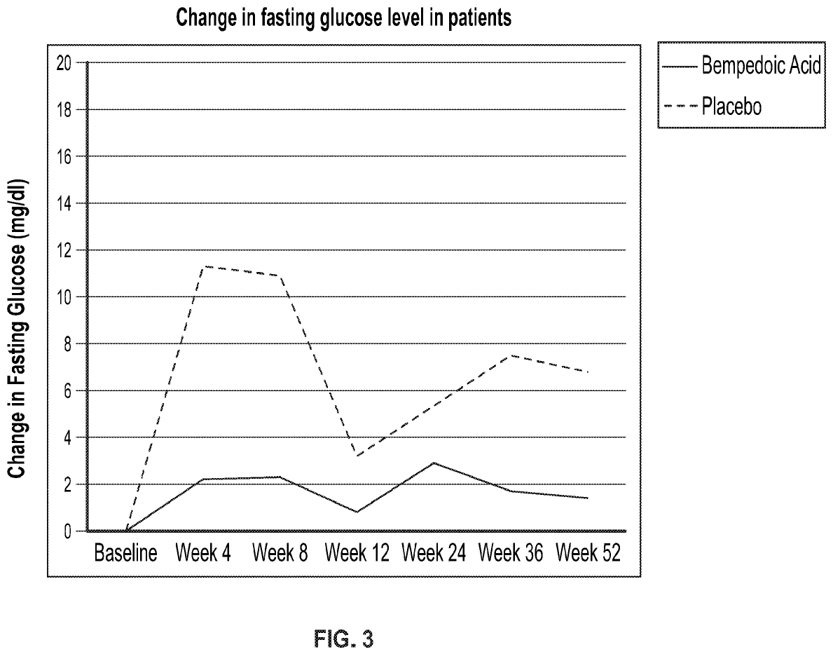

2230 total patients were receiving background MTD statin (50% high intensity) for 52 weeks. Of these, 637 patients (28.6%) had a medical history of diabetes. Patients were treated with bempedoic acid or placebo. The baseline HbA1c levels for patients receiving bempedoic acid was 6.85%, and the baseline HbA1c levels for patients receiving placebo was 6.89%. The baseline fasting glucose levels for patients receiving bempedoic acid was 131.4 mg/dl. The baseline fasting glucose levels for patients receiving placebo was 130.6 mg/dl. Changes in HbA1c levels were measured at week 12 and week 52 and changes in fasting glucose levels were measured at weeks 4, 8, 12, 24, and 36 and 52. The data are presented in FIG. 2 and FIG. 3 and demonstrate that bempedoic acid reduces the incidence of diabetes-related phenotypes, worsening of diabetes, and new onset of diabetes relative to placebo in patients receiving statin therapy.

### Example 2: Bempedoic Acid Reduces Diabetes Risk in Statin-Intolerant Patients

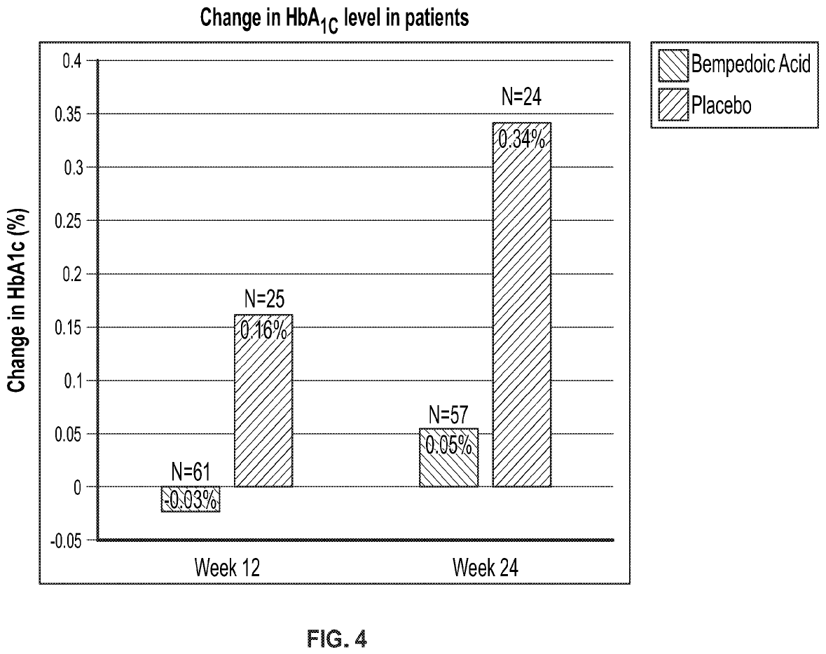

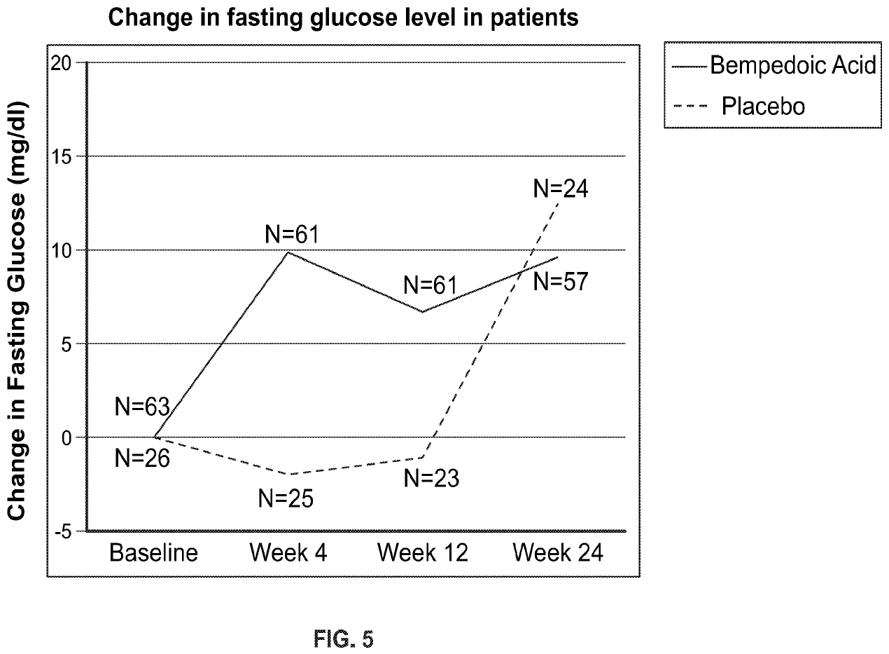

345 total patients with statin intolerance (8% very low dose statin) were treated for 24 weeks. Of these, 89 patients (25.8%) had a medical history of diabetes. Patients were treated with bempedoic acid or placebo. The baseline HbA1c levels for patients receiving bempedoic acid was 6.91%, and the baseline HbA1c levels for patients receiving placebo was 7.12%. The baseline fasting glucose levels for patients receiving bempedoic acid was 130.7 mg/dl. The baseline fasting glucose levels for patients receiving placebo was 139.8 mg/dl. Changes in HbA1c levels were measured at week 12 and week 24 and changes in fasting glucose levels were measured at weeks 4, 12, and 24. The data are presented in FIG. 4 and FIG. 5 and demonstrate that bempedoic acid reduces incidence of diabetes-related phenotypes, worsening of diabetes, and new onset of diabetes relative to placebo in statin intolerant patients.

### Example 3: Bempedoic Acid Reduces Diabetes Risk in Statin-Intolerant Patients on Background Ezetimibe Therapy

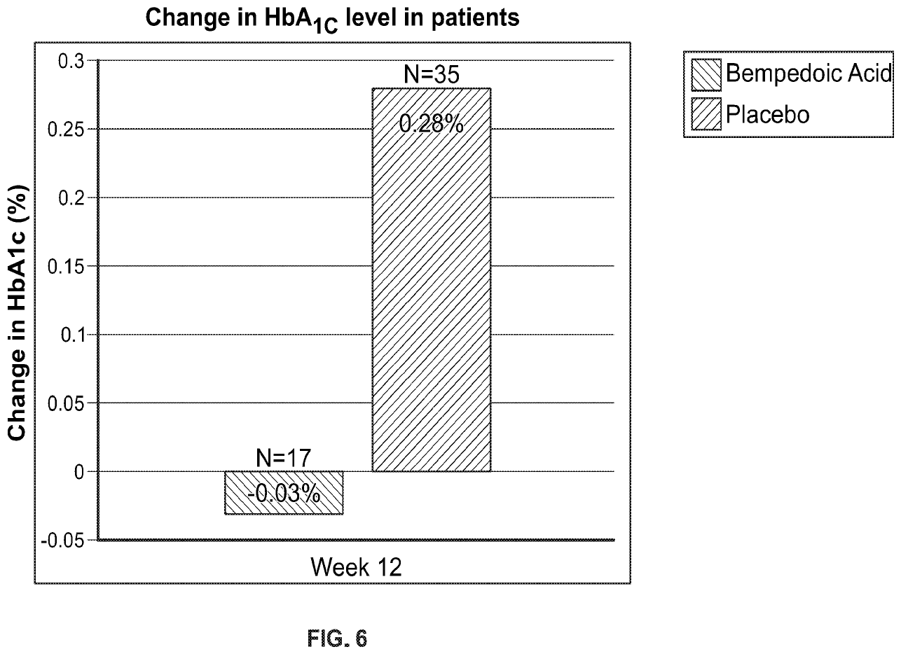

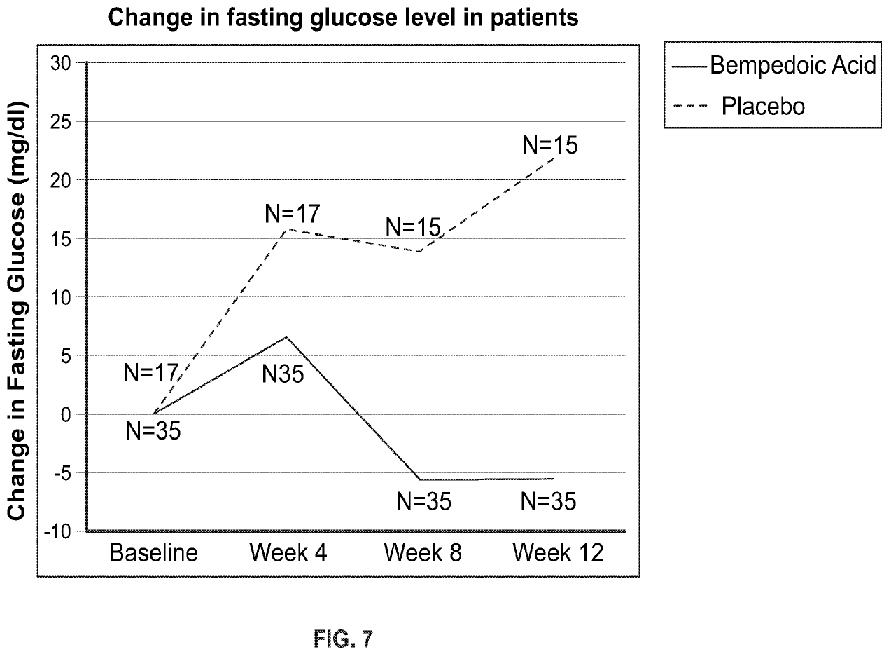

269 total patients with statin intolerance (31% low/very dose statin) were treated for 12 weeks; all patients on background ezetimibe therapy. Of these, 52 patients (19.3%) had a medical history of diabetes. Patients were treated with bempedoic acid or placebo. The baseline HbA1c levels for patients receiving bempedoic acid was 6.66%, and the baseline HbA1c levels for patients receiving placebo was 6.76%. The baseline fasting glucose levels for patients receiving bempedoic acid was 133.7 mg/dl. The baseline fasting glucose levels for patients receiving placebo was 133.4 mg/dl. Changes in HbA1c levels were measured at week 12, and changes in fasting glucose levels were measured at weeks 4, 8, and 12. The data are presented in FIG. 6 and FIG. 7 and demonstrate that bempedoic acid reduces the incidence of diabetes-related phenotypes, worsening of diabetes, and new onset of diabetes relative to placebo in statin intolerant patients on background ezetimibe therapy.

### Example 4: Bempedoic Acid+Ezetimibe Treatment Reduces Diabetes Risk in Patients Receiving Ezetimibe

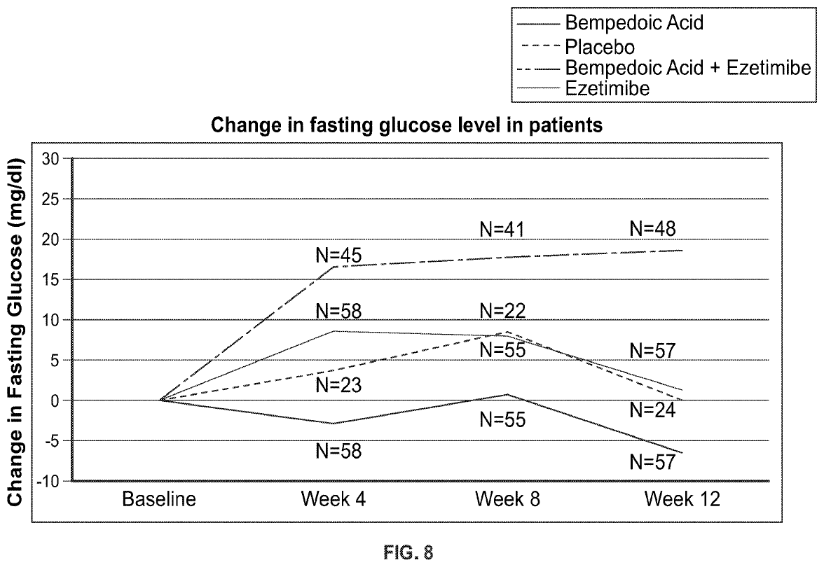

381 total patients on maximally tolerated station dosages (38.6% on high intensity statin; 28% on no statin) were treated for 12 weeks. Of these, 195 patients (51.2%) had a medical history of diabetes. Patients were randomized to 1 of 4 arms: bempedoic acid+ezetimibe; bempedoic acid; ezetimibe; or placebo (2:2:2:1). The baseline fasting glucose levels for patients receiving bempedoic acid+ezetimibe was 125.8 mg/dl. The baseline fasting glucose levels for patients receiving bempedoic acid was 130.9 mg/dl. The baseline fasting glucose levels for patients receiving ezetimibe was 142.5 mg/dl. The baseline fasting glucose levels for patients receiving placebo was 124.9 mg/dl. Fasting glucose levels were measured at weeks 4, 8, and 12. The data are presented in FIG. 8 and demonstrate that bempedoic acid+ezetimibe reduces the incidence of diabetes-related phenotypes, worsening of diabetes, and new onset of diabetes in patients receiving relatively high intensity statin therapy.

The cumulative data presented in these Examples demonstrates (among other findings) that new onset or worsening of diabetes and diabetes-related symptoms occurs less frequently in patients taking statins plus bempedoic acid (4%) relative to patients taking statins plus placebo (5.6%).

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B (1992).

Any terms not directly defined herein shall be understood to have the meanings commonly associated with them as understood within the art of the invention. Certain terms are discussed herein to provide additional guidance to the practitioner in describing the compositions, devices, methods and the like of aspects of the invention, and how to make or use them. It will be appreciated that the same thing may be said in more than one way. Consequently, alternative language and synonyms may be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples, including examples of terms, is for illustrative purposes only and does not limit the scope and meaning of the aspects of the invention herein.

It must be noted that, as used in the specification and the appended claims, the singular forms “a,” “an” and “the” include plural referents unless the context clearly dictates otherwise.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

What is claimed:

1. A method for decreasing the likelihood of new-onset diabetes in a subject or decreasing the likelihood of worsening existing diabetes in a subject comprising administering a fixed-dose of ETC-1002 or an analog thereof, a fixed-dose of ezetimibe or an analog thereof, and a fixed-dose of statin, to the subject.

2. The method of claim 1, wherein the subject has diabetes.

3. The method of claims 1 and 2, wherein said diabetes is selected from the group consisting of Type 1 diabetes, Type 2 diabetes, Type 3 diabetes, gestational diabetes, latent autoimmune diabetes in adults, maturity onset diabetes of the young, double diabetes, steroid-induced diabetes, brittle diabetes, secondary diabetes, diabetes insipidus, and Juvenile diabetes.

4. The method of claims 1 and 2, wherein the subject has a history of Type 2 diabetes for 6 months or greater.

5. The method of any one of claims 1-4, wherein the subject has been taking stable diabetes medication for three months.

6. The method of any one of claims 1-4, wherein the subject has a HbA_{1c} level between 5% and 10%, or between 7% and 10%.

7. The method of any one of claims 1-5, wherein the subject has a fasting calculated LDL-C level of greater than 70 mg/dL.

8. The method of any one of claims 1-6, wherein the subject has a fasting calculated LDL-C level of between 100 and 220 mg/dL.

9. The method of claim 7, wherein the subject has had a washout of all lipid-modifying therapy (LMT).

10. The method of any one of claims 1-8, wherein the subject is not pregnant.

11. The method of any one of claims 1-9, wherein the subject does not have a body mass index (BMI) of greater than 40 kg/m².

12. The method of any one of claims 1-10, wherein the subject does not have a history of documented cardiovascular disease.

13. The method of claim 11, wherein the cardiovascular condition is selected from the group consisting of myocardial infarction, severe or unstable angina pectoris, coronary angioplasty, coronary artery bypass graft, stroke, transient ischemic attack, cerebrovascular event, symptomatic carotid artery disease, and symptomatic peripheral arterial disease.

14. The method of claim 11, wherein the subject does not have uncontrolled hypertension.

15. The method of claim 13, wherein the uncontrolled hypertension is defined as comprising a mean systolic blood pressure of greater than or equal to 160 mmHg.

16. The method of claim 13, wherein the uncontrolled hypertension is defined as comprising a diastolic blood pressure of greater than or equal to 100 mmHg.

17. The method of any one of claims 1-15, wherein the subject does not have a fasting triglycerides of greater than 400 mg/dL.

18. The method of any one of claims 1-16, wherein the subject does not have a history of Type 1 diabetes.

19. The method of any one of claims 1-18, wherein the subject does not have uncontrolled hypothyroidism.

20. The method of claim 19, wherein the said uncontrolled hypothyroidism comprises a value for thyroid-stimulating hormone (TSH) of greater than 1.5 times the upper limit of normal (ULN).

21. The method of any one of claims 1-20, wherein the subject does not have liver disease or dysfunction.

22. The method of claim 21, wherein the said liver disease or dysfunction is selected from the group consisting of positive serology for hepatitis B surface antigen (HBsAg), hepatitis C virus antibodies (HCV-AB), serum alanine aminotransferase (ALT) or aspartate aminotransferase (AST) value of greater than or equal to two times the ULN, and serum total bilirubin (TB) value of greater than or equal to two times the ULN.

23. The method of any one of claims 1-22, wherein the subject does not have renal dysfunction or glomerulonephritis.

24. The method of claim 23, wherein the said renal dysfunction or glomerulonephritis comprises a glomerular filtration rate (eGFR) of less than 30 mL/min/1.73 m².

25. The method of any one of claims 1-24, wherein the subject does not have gastrointestinal conditions or has undergone procedures that affect drug absorption.

26. The method of any one of claims 1-25, wherein the subject does not have hematologic disorder.

27. The method of any one of claims 1-26, wherein the subject does not have coagulation disorder.

28. The method of any one of claims 1-27, wherein the subject does not have a hemoglobin (Hgb) level of less than 10.0 g/dL.

29. The method of any one of claims 1-28, wherein the subject does not have an active malignancy.

30. The method of claim 29, wherein the said active malignancy is selected from the group consisting of surgery, chemotherapy, and radiation therapy.

31. The method of any one of claims 1-30, wherein the subject does not have an unexplained serum creatine kinase (CK) value of greater than three times the ULN.

32. The method of any one of claims 1-31, wherein the subject does not have a history of drug or alcohol abuse.

33. The method of any one of claims 1-32, wherein the subject does not use amphetamine or its derivatives.

34. The method of any one of claims 1-33, wherein the subject has not donated blood, or undergone multiple blood draws within 30 days.

35. The method of any one of claims 1-34, wherein the subject has not received blood transfusion, or undergone surgery within 30 days.

36. The method of any one of claims 1-35, wherein the subject has not previously participated in a clinical trial study comprises of administration of bempedoic acid.

37. The method of any one of claims 1-36, wherein the subject does not have a history of intolerance to ezetimibe.

38. The method of any one of claims 1-37, wherein the subject has not used a prohibited drug selected from the group consisting of statins, fibrates, niacin, niacin derivatives, bile acid sequestants, ezetimibe, apheresis, mipomersen, lomitapide, proprotein convertase subtilisin/kexin type 9 (PCSK9) inhibitors, cholesteryl ester transfer protein (CETP) inhibitors, red yeast rice extract-containing products, Omega 3 fatty acids and derivatives, over-the-counter (OTC) fish oil, and systemic corticosteroids.

39. The method of claim 1, wherein the subject has risk factors for Type 2 diabetes.

40. The method of claim 39, wherein the said risk factors is selected from the group consisting of overweight or obesity, 45 years of age or older, family history of diabetes, high blood pressure, a low level of high-density lipoprotien cholesterol, a high level of triglycerides, a history of gestational diabetes, heart disease or stroke, polycystic ovary syndrome, and acanthosis nigricans.

41. The method of claim 1, wherein the subject is receiving a maximally tolerated statin therapy.

42. The method of any one of claims 1-41, wherein ETC-1002 is administered at a fixed dose of about 120 milligram.

43. The method of any one of claims 1-41, wherein ETC-1002 is administered at a fixed dose of about 180 milligram.

44. The method of any one of claims 1-43, wherein ezetimibe is administered at a fixed dose of about 10 milligram.

45. The method of any one of claims 1-44, wherein the subject experiences a statistically significant change in LDL-C level from baseline after 12 weeks following receiving a fixed dose of ETC-1002 of about 120 mg or 180 mg and a fixed dose of ezetimibe of about 10 mg.

46. The method of claim 45, wherein the subject is receiving a maximally tolerated statin therapy.

47. The method of any one of claims 1-45, wherein the subject experiences a statistically significant change in LDL-C level from baseline after 24 weeks following receiving a fixed dose of ETC-1002 of about 120 mg or 180 mg and a fixed dose of ezetimibe of about 10 mg.

48. The method of claim 47, wherein the subject is receiving a maximally tolerated statin therapy.

49. The method of any one of claims 1-46, wherein the subject experiences a statistically significant change in non-LDL-C, total cholesterol, apoprotein B, or C-reactive protein level from baseline after 12 weeks following receiving a fixed dose of ETC-1002 of about 120 mg or 180 mg while and a fixed dose of ezetimibe of about 10 mg

50. The method of 49, wherein the subject is receiving a maximally tolerated statin therapy.

51. The method of any one of claims 1-47, wherein the subject experiences a statistically significant improvement in HbA_{1C} level from baseline after 12 weeks following receiving fixed dose of ETC-1002 of about 120 mg and fixed dose of ezetimibe of about 10 mg.

52. The method of claim 51, wherein the subject is receiving a maximally tolerated statin therapy.

53. The method of any one of claims 1-52, wherein the subject is a human.

54. The method of claim 53, wherein the method comprises identifying the subject as a subject with diabetes or with an increased risk of diabetes relative to normal, and further comprising administering to said subject an amount of bempedoic acid, or ezetimibe, or a combination of bempedoic acid and ezetimibe.

55. The method of claim 54, wherein the amount of bempedoic acid or ezetimibe or both is effective to reduce LDL-C in said subject.

56. The method of claim 54 or 55, wherein the increased risk of diabetes is the result of ongoing or expected statin therapy.
